(19) United States
(12) Patent Application Publication
(10) Pub. No.: US 2020/0147333 A1
(43) Pub. Date: May 14, 2020

(54) BREATH SAMPLE SYSTEMS FOR USE WITH VENTILATORS

(71) Applicant: Hound Labs, Inc., Oakland, CA (US)

(72) Inventors: Jeffrey A. Stoll, San Mateo, CA (US); Steven Joseph Duquette, Laguna Niguel, CA (US)

(21) Appl. No.: 16/676,562

(22) Filed: Nov. 7, 2019

Related U.S. Application Data
(60) Provisional application No. 62/758,329, filed on Nov. 9, 2018.

(51) Int. Cl. A61M 16/08; A61B 10/00; A61M 16/20; A61B 5/00

(52) U.S. Cl. CPC A61M 16/0841; A61B 10/00; A61M 16/0858; A61B 5/097; A61M 16/20; A61B 5/7285; A61B 2010/0087; A61M 16/0883

(57) ABSTRACT

Breath sample systems for use with ventilators are disclosed herein. An example device can include a housing that contains a breath capture module that collects a breath sample from a patient, an input in fluid communication with an expiratory limb of a ventilator and the breath capture module, the input receiving a breath of the patient, an output in fluid communication with the expiratory limb of a ventilator to return the breath to the ventilator, a pump, and a controller that controls the pump to obtain the breath sample by drawing the breath into the input, across the breath capture module, and out of the output.

### CROSS REFERENCE TO RELATED APPLICATIONS

This application claims the benefit and priority of U.S. Provisional Application Ser. No. 62/758,329 filed on Nov. 9, 2018 and entitled “Device to Sample Non-Volatile Biomarkers in Exhaled Breath”. The disclosure of the above-referenced application is hereby incorporated by reference herein in its entirety, including all references and appendices cited therein, for all purposes.

### FIELD OF THE INVENTION

The present disclosure is directed generally to breath sample cartridge systems and methods, which can be used in combination with a ventilator system to obtain breath samples from a patient.

### SUMMARY

Some embodiments of the present disclosure can be directed to a device for use with a ventilator, the device comprising a housing that includes a breath capture module that collects a breath sample from a patient, an input in fluid communication with an expiratory limb of a ventilator and the breath capture module, the input receiving a breath of the patient, an output in fluid communication with the expiratory limb of a ventilator to return the breath to the ventilator, a pump, and a controller that controls the pump to obtain the breath sample by drawing the breath into the input, across the breath capture module, and out of the output.

Some embodiments of the present disclosure can be directed to a system comprising a ventilator having an inspiratory limb and an expiratory limb, a breath capture device coupled with the expiratory limb of the ventilator, the breath capture device comprising, a breath capture module comprising a pump; and a controller comprising a processor and memory for storing instructions, the processor executing the instructions to control the pump to obtain breath from the expiratory limb of the ventilator and return the breath to the expiratory limb after the breath has contacted the breath capture module; the breath capture module collecting a breath sample from the breath.

Some embodiments of the present disclosure can be directed to a method comprising determining a flowrate or periodicity of an expiratory phase of a ventilator and controlling a pump in response to the flowrate or periodicity to obtain a breath sample, wherein the breath sample is obtained by directing breath from an expiratory limb of the ventilator through a breath capture module and returning the breath to the expiratory limb.

### BRIEF DESCRIPTION OF THE DRAWINGS

The accompanying drawings, where like reference numerals refer to identical or functionally similar elements throughout the separate views, together with the detailed description below, are incorporated in and form part of the specification, and serve to further illustrate embodiments of concepts that include the claimed disclosure, and explain various principles and advantages of those embodiments.

The methods and systems disclosed herein have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

FIG. 1 illustrates an environment where aspects of the present disclosure can be practiced, along with a schematic diagram of a ventilator in combination with an example breath capture device of the present disclosure.

FIG. 2 is a flowchart of an example method for breath sampling using an example breath capture device of the present disclosure.

FIG. 3 is a flowchart of another example method for breath sampling using an example breath capture device of the present disclosure.

FIG. 4 is a flowchart of a further example method for breath sampling using an example breath capture device of the present disclosure.

### DETAILED DESCRIPTION

Generally speaking, the present disclosure relates to devices that sample biomarkers in a subject's exhaled breath. One embodiment of a breath capture device of the present disclosure captures aerosolized particles that originate in the deep lung and pass through upper airways and out the mouth with exhalation. Composed mainly of airway lining fluid and water, these aerosolized droplets also contain proteins and other biomarkers of interest. After capture, the captured droplets can be processed chemically to isolate compounds of interest. Such devices have been tested extensively with human subjects for detection of drugs of abuse, with subjects exhaling through a mouthpiece into the device. In some implementations, described herein, an example breath capture device can be adapted to sample the exhaled breath from ventilated subjects/patients.

An example device to sample non-volatile biomarkers in a subject's exhaled breath can be integrated with a ventilator to allow sampling of exhaled breath without interfering with the normal operation of the ventilator. A breath sampling device can be connected to the proximal end of the expiratory limb of the ventilator's patient circuit, and the return line connected at the ventilator before an expiratory filter of the ventilator. This configuration can ensure that gas removed from the patient circuit directed through the breath sampling device is returned to the ventilator. It can also ensure that the breath sample collected by the device will comprise exhaled gas and not baseline flow being delivered to the patient by the ventilator. Returning the gas removed from the expiratory limb may reduce or avoid false/positive alarms relating to volume, inspiratory pressure, positive-end expiratory pressure (PEEP) sensing, and circuit disconnect and/or occlusion detection.

Integration of a breath sampling device of the present disclosure to periodically sample the exhaled breath path can ensure that the ventilator functions normally. In this way, the operation of the breath sample device can be both effective to collect biomarkers in a ventilated subject's exhaled breath for evaluation that may have a medical or diagnostic correlation of interest, while at the same time being transparent to clinical staff, thereby enhancing both utility and patient safety.

Downstream sample analysis may be conducted by a variety of techniques including fluorescent small molecule-based, immunoassay-based or mass spectrometry-based approaches, as generally understood and/or described in the materials incorporated by reference above. Suitable approaches may be selected based on technical and/or market requirements. Sensitivity of an assay method is often a driver. In some ventilator-integrated embodiments sampling time is not a limitation. If subjects/patients are exhaling very small amounts of a particular bio-marker or analyte, longer collection times can be used. Further, depending on technical or market needs, discrete or continuous sampling can be used. For example, if a market needs qualitative measurements tracked continuously over time, an immunoassay method with point-of-care results may be preferred. If desired, a single sample can be analyzed on a longer sample-to-answer timeline, allowing for more straightforward mass spectrometry.

In sum, devices and methods described herein may be implemented in ventilator-integrated systems for breath sampling to collect biomarkers in a ventilated subject's exhaled breath for evaluations that may have medical or diagnostic correlations of interest, among other uses.

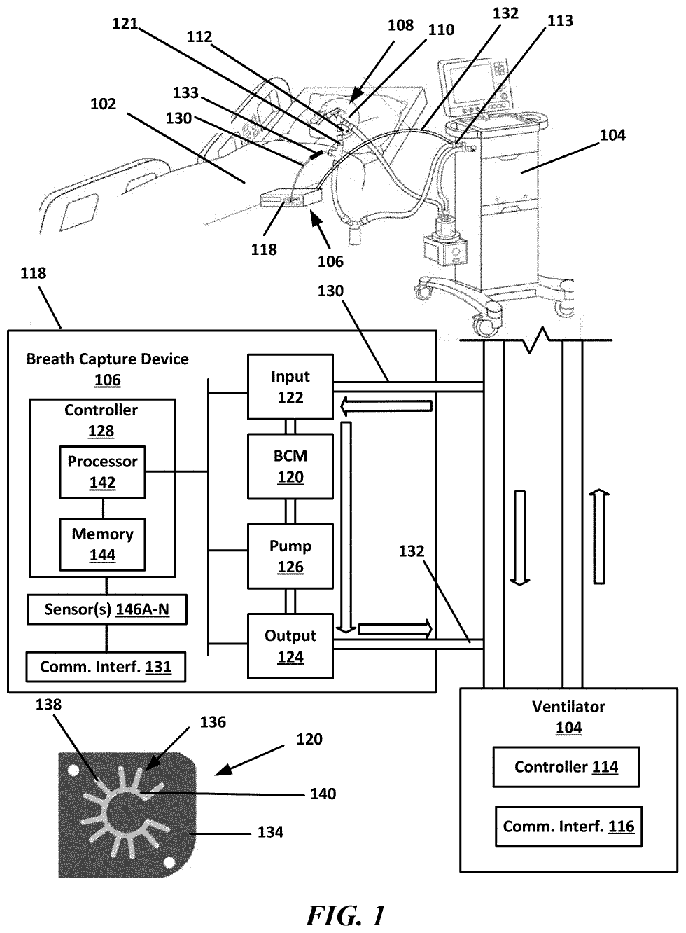

FIG. 1 illustrates an example environment for practicing aspects of the present disclosure. The environment includes a patient 102, a ventilator 104, and a breath capture device 106. In general, the patient is in an intubated state, through use of a delivery catheter (e.g., endotracheal tube) inserted into a mouth of the patient or coupled with a tracheostomy circuit (not shown).

The ventilator 104 is configured to mechanically respire for the patient 102. In general, mechanical respiration involves an inspiratory phase where air is pushed into the lungs of the patient 102, as well as an expiratory phase when air exits the patient 102 through extraction. In some embodiments, the ventilator 104 is coupled to the patient 102 with a ventilator circuit 108 that includes an inspiratory limb 110 and an expiratory limb 112.

The ventilator 104 can comprise a controller 114 that is configured to control inspiratory and expiratory phases. For example, the controller 114 can alternate between inspiratory and expiratory phases to mechanically respire for the patient 102. In various embodiments, the ventilator 104 comprises a communications interface 116 that allows an external device, such as the breath capture device 106 to communicatively couple with the ventilator 104. Various ventilator data can be obtained through the communications interface 116 such as an electrical trigger signal that is indicative of the inspiratory and expiratory phases. Generally, the electrical trigger signal is indicative of an operational voltage of the ventilator 104. The operational voltage can vary from a first voltage value that is indicative of an inspiratory phase and a second voltage value that is indicative of an expiratory phase. In one example, the first voltage value could include zero voltage and the second voltage value could include five volts. To be sure, these are example or reference voltage values and are not intended to be limiting. Typically, the exact magnitudes of the voltage values vary according to the operating parameters of the ventilator 104.

In some embodiments, the controller 114 can be configured to maintain inspiratory and expiratory phases of twelve to sixteen breaths per minute. An example inspiratory phase may drive a bolus of approximately 500-750 milliliters of air into the patient 102 and enforce expiratory phases where the bolus is removed over a period of time of approximately 300 milliseconds.

Broadly speaking, the breath capture device 106 comprises a housing 118, a breath capture module (BCM 120), an input 122, an output 124, a pump 126, a controller 128, and a communications interface 131. In some embodiments, the breath capture device 106 is coupled with the expiratory limb 112 of the ventilator circuit 108. For example, a first conduit 130 can fluidly couple the input 122 of the breath capture device 106 with the expiratory limb 112. In one embodiment, a T-shaped conduit 121 is installed in-line with the expiratory limb 112. The first conduit 130 is coupled to the T-shaped conduit 121. In various embodiments, the input 122 and output 124 can include a connection port. In other embodiments, the input 122 and output 124 include connection ports in combination with valves. For example, the input 122 could comprise a one-way valve that allows fluid to flow into the breath capture device 106, but not pass back through into the expiratory limb 112. The valve associated with the input 122 could be a controllable multi-way valve that can open and close as needed.

The breath capture device 106 can over the communications interface 131 with the ventilator 104 through its communications interface 116. An suitable communications protocol can be used, including any wired or wireless communications such as Wi-Fi, NFC (Near-Field Communications), Bluetooth, cellular, and so forth.

In various embodiments, a second conduit 132 is used to fluidly couple the output 124 with the expiratory limb 112. Generally, the output 124 is coupled to the expiratory limb 112 (through the second conduit 132) at a location that is downstream of a location where the input 122 is coupled with the expiratory limb 112 (through the first conduit 130). In one example, the second conduit 132 can couple to the expiratory limb 112 near an expiratory limb return port 113 of the ventilator 104.

In some embodiments, the input 122, BCM 120, pump 126, and output 124 are fluidly coupled to one another through a series of conduits within the housing 118. This fluid connectivity allows the pump to draw breath into the input 122, through the BCM 120, and out of the output 124, so as to capture breath samples in the BCM 120.

The breath capture module 120 can include a cartridge that can be installed, removed, and replaced in some instances. In exemplary embodiments, the cartridge may be a single use or multi-use disposable cartridge. The breath capture module 120 can comprise a body 134 that includes a capture media 136. The body 134 includes impaction ports 138 that allow breath to pass through the capture media 136 where aerosolized droplets are captured from the breath of the patient 102 to obtain a breath sample. Processed breath can be removed through exhaust ports 140 and ultimately through the output 124.

Aerosolized droplets may be captured from the breath of the patient 102 to obtain the breath sample through any number of means. Capture media 136 depicts one exemplary embodiment of a mechanical impactor mechanism for capturing aerosolized droplets from breath. However, alternative means for capturing breath may be used in other embodiments within the scope of this disclosure. For example, a plurality of capture media 136 may be present in the breath capture module 120 in series, to maximize the amount of aerosolized particles captured from each breath.

Alternatively, capture media 136 may take a different form than depicted in FIG. 1, such as one or more types of filters, beads, packed beds, micro-particles, or other mechanisms for capturing specific components from human breath.

In some embodiments of the present disclosure, a plurality of breath samples are obtained. The number of breath samples required can be based on the flow rate and/or periodicity of the ventilator 104. In some embodiments, the number of breath samples can include a threshold number that is based on empirical observation. Also, the number of breaths sampled can be further correlated to the operation of the pump 126 within the breath capture device 106. That is, the periodicity and/or flow rate of the breaths that contact the plurality of capture media 136 are controlled by the operation of the pump 126. Flow rate can be changed by increasing the output of the pump 126, whereas periodicity can be selected by cycling of the pump 126 on and off by the controller 128. In various embodiments, an ancillary flow augmentation device 133, such as an additional flow regulator can be added upstream of the breath capture device 106, which can be used to selectively control a flow rate into the breath capture device 106. Additionally, the ancillary flow augmentation device 133 could also include other components such as a secondary pump can be added upstream of the breath capture device 106, such as near the t-shaped conduit 121. This secondary pump can be used to pull breath into the first conduit 130 from the expiratory limb 112.

That is, in order to obtain a sufficient amount of aerosolized droplets, which would allow for one or more types of chemical analysis, multiple breath samples may be required. The number of breath samples obtained can be based on the flow rate, flow volume, flow pressure, and/or periodicity of the ventilator 104. For example, as noted above, the flow volume of the ventilator 104 may be approximately 500-750 milliliters of air and the expiratory phase may be approximately 300 milliseconds in duration. Also, it will be appreciated that only a portion of the breath passing through the expiratory limb 112 may enter the input 122 of the breath capture device 106. In some instances, the controller 128 of the breath capture device 106 can be configured to determine the flow rate, flow volume, flow pressure, and/or periodicity of the ventilator 104. As noted above, the controller 128 can obtain flow rate, flow volume, flow pressure, and/or periodicity of the ventilator 104 from ventilator operational data provided through the communications interface 116.

In various embodiments, the breath capture device 106 can comprise one or more sensors 146A-N, which can determine any of flow rate, flow volume, flow pressure, and/or periodicity of the expiratory flow through the expiratory limb 122 of the ventilator circuit 108. The one or more sensors 146A-N can be used to detect an amount of exhaled breath that is obtained from the expiratory limb 112 of the ventilator circuit 108.

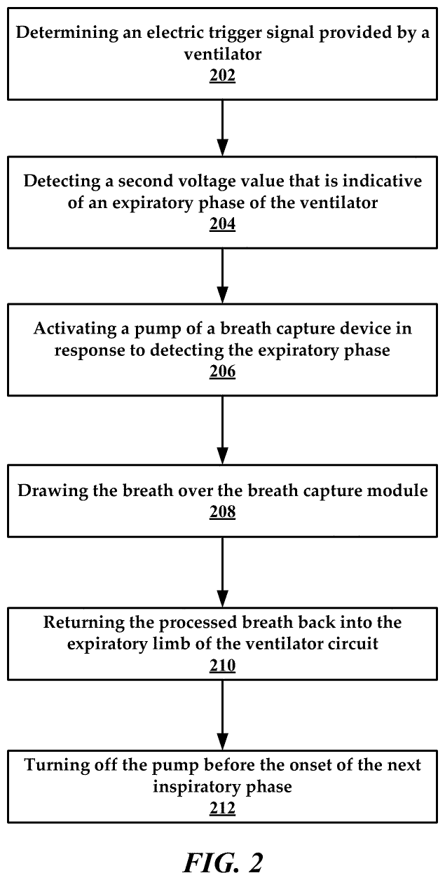

The controller 128 can comprise a processor 142 and memory 144. The processor 142 executes instructions stored in memory 144. The controller 128 can be configured to utilize ventilator operational data to control operation of the pump 126. Various processes for controlling the pump to obtain breath samples using the breath capture device 106 are illustrated in FIGS. 2-4. As noted above, the breath capture device 106 can be configured to obtain a plurality of breath samples from a plurality of expiratory phases. That is, in order to obtain a sufficient sample of aerosolized content on the breath capture module 120, the breath of multiple expiratory phases may be captured. Thus, the controller 128 can be configured to obtain a number of breath samples in some embodiments. The number of breath samples needed may be based on a threshold value. For example, some embodiments may involve processing a total of fifty to seventy liters of exhaled breath. If each bolus provided by the ventilator is 500 to 750 milliliters, the number of breath samples would range from 25 to 35 distinct samples when the flow amount is 500 milliliters. The desired volume of breath to be captured can vary based on the specific biomarker(s) to be analyzed from the captured breath.

According to some embodiments, the controller 128 can be configured to selectively control the pump 126 to turn on in response to detection of a beginning of the expiratory phase. The controller 128 can be configured to selectively control the pump 126 to turn off in response to the detection of a beginning of the inspiratory phase or an end of the expiratory phase. Again, the end of the expiratory phase can be determined based on a change in voltage or based on a counted time period. For example, each expiratory phase could be of a given duration (based on the specifics of each ventilator).

In some embodiments, the controller 128 can be configured to delay the activation of the pump 126 for a period of time at the beginning of an expiratory phase. For example, when the controller 128 determines than an expiratory phase has been initiated, the controller 128 can delay pump until a set period of time, such as 100 milliseconds after the initiation of the expiratory phase. This may ensure that what is sampled from the expiratory limb 112 of the ventilator circuit 108 is exhaled breath and not inspired flow or other fluid that may accumulate inside the expiratory limb 112 during an inspiratory phase.

In various embodiments, the controller 128 can be configured to open and/or close either the input 122 and/or the output 124 (as needed). For example, a valve associated with the input 122 and a valve associated with the output 124 can be closed. In some embodiments, the valves can include manual one-way-valves, or multi-way valves that can be selectively controlled using the controller 128.

When closed, a user can remove and replace the BCM 120 with a new BCM. This can allow a user to obtain a plurality of breath samples for various purposes. For example, the user could obtain a first set of BCMs to test for the presence of a first chemical, such as THC (Tetrahydrocannabinol). The user could obtain a second set of BCMs to test for the presence of a second chemical, such as Ethanol. While these two specific chemicals are listed here for exemplary purposes, it is to be understood that the embodiments disclosed herein can be used to test for the presence of any number of chemicals found in human breath.

FIG. 2 illustrates an example control method for obtaining a breath sample. The method involves periodic sampling of patient breath using electrical trigger signal provided by a ventilator. Thus, the method includes a step 202 of determining an electrical trigger signal provided by a ventilator. As noted above, the electrical trigger signal can be determined by coupling with a communications interface of the ventilator and sensing changes in an operational voltage of the ventilator. To be sure, the operational voltage can vary from a first voltage value that is indicative of an inspiratory phase and a second voltage value that is indicative of an expiratory phase. In one example, the first voltage value could include zero voltage and the second voltage value could include five volts. To be sure, these are example or reference voltage values and are not intended to be limiting. Typically, the exact magnitudes of the voltage values vary according to the operating parameters of the ventilator 104.

For example, the method includes a step 204 of detecting second voltage value that is indicative of an expiratory phase of the ventilator, along with a step 206 of activating a pump of a breath capture device in response. In one example, this can include detecting when the second voltage value is approximately zero.

The activation of the pump draws exhaled breath from the expiratory limb of the ventilator circuit into the breath capture device so that the breath contacts a breath capture module, thereby capturing a breath sample. Thus, the method includes a step 208 of drawing the breath over the breath capture module.

Activation of the pump also results in recirculation of the processed breath back into the expiratory limb of the ventilator circuit. The flow rate of the pump is selected to maintain a predetermined configurable flow rate of the diverted breath into and out of the breath capture device, as well as back into the expiratory limb of the ventilator circuit. Thus, the method includes a step 210 of returning the processed breath back into the expiratory limb of the ventilator circuit.

In general, mechanical ventilators may provide an electrical trigger signal. Breath sampling by the breath capture device can be triggered by this signal from the ventilator. Ventilators may have a zero to five volt signal for the start of inspiration and exhalation (five volts of direct current is correlated with inspiration, zero volts of direct current being correlated with exhalation). Sampling of exhaled gas can be initiated when the ventilator output signal is approximately zero Volts, and continuing for a predetermined time, after which the pump can be turned off. As noted above, such periodic sampling can ensure that the sample being taken is exhaled flow from the patient and not baseline flow being delivered by the ventilator. Turning the pump off before the onset of the next inspiratory phase allows the ventilator's trigger algorithm to function normally. Thus, the method can include a step 212 of turning off the pump before the onset of the next inspiratory phase. This could occur when the controller of the breath capture device detects an increase in voltage from zero Volts or at the expiration of a period of time (e.g., based on periodicity). Thus, if the ventilator operates at regularly timed intervals, the breath capture device can detect changes in flowrate through the expiratory limb and determine periodicity.

Also, it will be understood that returning the processed breath to the ventilator should avoid false/positive alarms relating to volume, inspiratory pressure, positive-end expiratory pressure (PEEP) sensing, and circuit disconnect and/or occlusion detection.

In general, the method includes steps involving controlling a pump in response to the flowrate or periodicity to obtain a breath sample. To be sure, periodicity can be determined from voltage changes indicated by the electrical trigger signal produced by the ventilator.

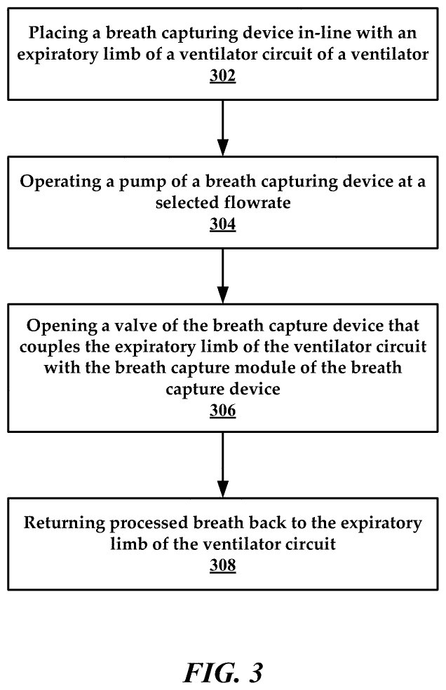

FIG. 3 illustrates a flowchart of another example method for obtaining a breath sample from an expiratory limb of a ventilator circuit. For context, some ventilators operate at relatively short inspiratory and/or expiratory phases. That is, the average exhalation duration for ventilated subjects may be regulated by the ventilator. Individuals typically respire at a rate of four to six breaths per minute in comparison with the 12-16 breaths that are used for ventilator patients. Typical lung-protective strategies in ventilated patients may drive 500-750 milliliters of air into a patient and enforce exhalation times of about 300 milliseconds. Since these are faster timeframes and lower volumes per breath cycle, there is a potential for missing sample as the particle-laden flow goes through the ventilator (e.g., a 750 milliliters of breath bolus passing through the ventilator in a 300 milliseconds exhalation duration could be missed in the time it takes for the breath sample device pump to reach peak flow rate).

As such, a different strategy for sample collection may be implemented in some ventilator-integrated embodiments. In some such embodiments, continuously running the pump in the breath sample device and relying on a fast-response solenoid valve to shuttle a particle-laden sample to the impaction subsystem may provide a more consistent and reliable sampling of breath.

The method can include a step 302 of placing a breath capturing device in-line with an expiratory limb of a ventilator circuit of a ventilator. This can include installing a fitting in the expiratory limb and coupling an input of the breath capturing device to the fitting using a first conduit such as a hose. A second conduit is used to couple the output of the breath capturing device to the expiratory limb at a location that is downstream and proximate to the ventilator. For example, the second conduit can be connected near an expiratory limb return port of the ventilator.

The method can include a step 304 of operating a pump of a breath capturing device at a selected flow rate. Again, the flowrate can be approximately matched to the flowrate of the ventilator, which can be determined based on the ventilator operational data obtained from the ventilator. The initialization of the pump may be triggered when the breath sample device is installed in-line with the expiratory limb and communicatively coupled with the ventilator.

The method can include a step 306 of opening a valve of the breath capture device that couples the expiratory limb of the ventilator circuit with the breath capture module of the breath capture device. The valve can include a fast-response solenoid valve. The controller of the breath capture device can toggle the valve between open and closed positions, allowing the pump to run in a continuous manner. This allows the breath capture device to be responsive to expiratory periods of time that are relatively short for some ventilators. The method can include a step 308 of returning processed breath back to the expiratory limb of the ventilator circuit.

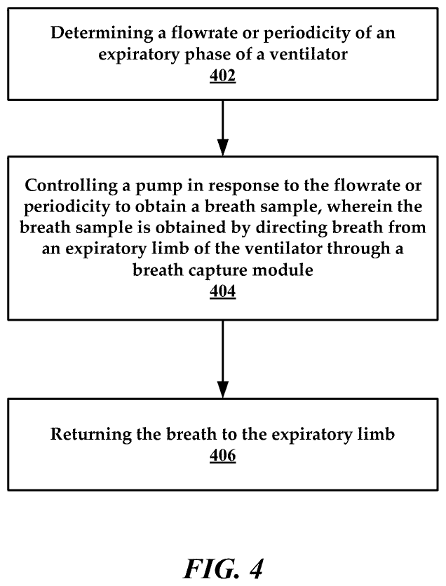

FIG. 4 illustrates another example flowchart of a method for controlling a breath capture device to obtain a breath sample. The method can include a step 402 of determining a flowrate or periodicity of an expiratory phase of a ventilator. The method can also include a step 404 of controlling a pump in response to the flowrate or periodicity to obtain a breath sample. To be sure, the breath sample is obtained by directing breath from an expiratory limb of the ventilator through a breath capture module. The method further includes a step 406 of returning the breath to the expiratory limb.

In the above disclosure, reference has been made to the accompanying drawings, which form a part hereof, which illustrate specific implementations in which the present disclosure may be practiced. It is understood that other implementations may be utilized, and structural changes may be made without departing from the scope of the present disclosure. References in the specification to “one embodiment,” “an embodiment,” “an example embodiment,” and the like indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, one skilled in the art will recognize such feature, structure, or characteristic in connection with other embodiments whether or not explicitly described.

Implementations of the systems, apparatuses, devices, and methods disclosed herein may comprise or utilize a special purpose or general-purpose computer including computer hardware, such as, for example, one or more processors and system memory, as discussed herein. Implementations within the scope of the present disclosure may also include physical and other computer-readable media for carrying or storing computer-executable instructions and/or data structures. Such computer-readable media can be any available media that can be accessed by a general-purpose or special purpose computer system. Computer-readable media that stores computer-executable instructions is computer storage media (devices). Computer-readable media that carries computer-executable instructions is transmission media. Thus, by way of example, and not limitation, implementations of the present disclosure can comprise at least two distinctly different kinds of computer-readable media: computer storage media (devices) and transmission media.

Computer storage media (devices) includes RAM, ROM, EEPROM, CD-ROM, solid state drives (SSDs) (e.g., based on RAM), flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

An implementation of the devices, systems, and methods disclosed herein may communicate over a computer network. A “network” is defined as one or more data links that enable the transport of electronic data between computer systems and/or modules and/or other electronic devices. When information is transferred or provided over a network or another communications connection (either hardwired, wireless, or any combination of hardwired or wireless) to a computer, the computer properly views the connection as a transmission medium. Transmission media can include a network and/or data links, which can be used to carry desired program code means in the form of computer-executable instructions or data structures and which can be accessed by a general purpose or special purpose computer. Combinations of the above should also be included within the scope of computer-readable media.

Computer-executable instructions comprise, for example, instructions and data which, when executed at a processor, cause a general purpose computer, special purpose computer, or special purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the present disclosure may be practiced in network computing environments with many types of computer system configurations, including in-dash vehicle computers, personal computers, desktop computers, laptop computers, message processors, handheld devices, multi-processor systems, microprocessor-based or programmable consumer electronics, network PCs, minicomputers, mainframe computers, mobile telephones, PDAs, tablets, pagers, routers, switches, various storage devices, and the like. The disclosure may also be practiced in distributed system environments where local and remote computer systems, which are linked (either by hardwired data links, wireless data links, or by any combination of hardwired and wireless data links) through a network, both perform tasks. In a distributed system environment, program modules may be located in both the local and remote memory storage devices.

Further, where appropriate, the functions described herein can be performed in one or more of hardware, software, firmware, digital components, or analog components. For example, one or more application specific integrated circuits (ASICs) can be programmed to carry out one or more of the systems and procedures described herein. Certain terms are used throughout the description and claims refer to particular system components. As one skilled in the art will appreciate, components may be referred to by different names. This document does not intend to distinguish between components that differ in name, but not function.

It should be noted that the sensor embodiments discussed above may comprise computer hardware, software, firmware, or any combination thereof to perform at least a portion of their functions. For example, a sensor may include computer code configured to be executed in one or more processors and may include hardware logic/electrical circuitry controlled by the computer code. These example devices are provided herein for purposes of illustration and are not intended to be limiting. Embodiments of the present disclosure may be implemented in further types of devices, as would be known to persons skilled in the relevant art(s).

At least some embodiments of the present disclosure have been directed to computer program products comprising such logic (e.g., in the form of software) stored on any computer-usable medium. Such software, when executed in one or more data processing devices, causes a device to operate as described herein.

If any disclosures are incorporated herein by reference and such incorporated disclosures conflict in part and/or in whole with the present disclosure, then to the extent of conflict, and/or broader disclosure, and/or broader definition of terms, the present disclosure controls. If such incorporated disclosures conflict in part and/or in whole with one another, then to the extent of conflict, the later-dated disclosure controls.

The terminology used herein can imply direct or indirect, full or partial, temporary or permanent, immediate or delayed, synchronous or asynchronous, action or inaction. For example, when an element is referred to as being “on,” “connected” or “coupled” to another element, then the element can be directly on, connected or coupled to the other element and/or intervening elements may be present, including indirect and/or direct variants. In contrast, when an element is referred to as being “directly connected” or “directly coupled” to another element, there are no intervening elements present.

Although the terms first, second, etc. may be used herein to describe various elements, components, regions, layers and/or sections, these elements, components, regions, layers and/or sections should not necessarily be limited by such terms. These terms are only used to distinguish one element, component, region, layer or section from another element, component, region, layer or section. Thus, a first element, component, region, layer or section discussed below could be termed a second element, component, region, layer or section without departing from the teachings of the present disclosure.

The terminology used herein is for the purpose of describing particular embodiments only and is not intended to be necessarily limiting of the disclosure. As used herein, the singular forms “a,” “an” and “the” are intended to include the plural forms as well, unless the context clearly indicates otherwise. The terms “comprises,” “includes” and/or “comprising,” “including” when used in this specification, specify the presence of stated features, integers, steps, operations, elements, and/or components, but do not preclude the presence or addition of one or more other features, integers, steps, operations, elements, components, and/or groups thereof.

Example embodiments of the present disclosure are described herein with reference to illustrations of idealized embodiments (and intermediate structures) of the present disclosure. As such, variations from the shapes of the illustrations as a result, for example, of manufacturing techniques and/or tolerances, are to be expected. Thus, the example embodiments of the present disclosure should not be construed as necessarily limited to the particular shapes of regions illustrated herein, but are to include deviations in shapes that result, for example, from manufacturing.

Any and/or all elements, as disclosed herein, can be formed from a same, structurally continuous piece, such as being unitary, and/or be separately manufactured and/or connected, such as being an assembly and/or modules. Any and/or all elements, as disclosed herein, can be manufactured via any manufacturing processes, whether additive manufacturing, subtractive manufacturing and/or other any other types of manufacturing. For example, some manufacturing processes include three dimensional (3D) printing, laser cutting, computer numerical control (CNC) routing, milling, pressing, stamping, vacuum forming, hydroforming, injection molding, lithography and/or others.

Any and/or all elements, as disclosed herein, can include, whether partially and/or fully, a solid, including a metal, a mineral, a ceramic, an amorphous solid, such as glass, a glass ceramic, an organic solid, such as wood and/or a polymer, such as rubber, a composite material, a semiconductor, a nano-material, a biomaterial and/or any combinations thereof. Any and/or all elements, as disclosed herein, can include, whether partially and/or fully, a coating, including an informational coating, such as ink, an adhesive coating, a melt-adhesive coating, such as vacuum seal and/or heat seal, a release coating, such as tape liner, a low surface energy coating, an optical coating, such as for tint, color, hue, saturation, tone, shade, transparency, translucency, non-transparency, luminescence, anti-reflection and/or holographic, a photo-sensitive coating, an electronic and/or thermal property coating, such as for passivity, insulation, resistance or conduction, a magnetic coating, a water-resistant and/or waterproof coating, a scent coating and/or any combinations thereof.

Unless otherwise defined, all terms (including technical and scientific terms) used herein have the same meaning as commonly understood by one of ordinary skill in the art to which this disclosure belongs. The terms, such as those defined in commonly used dictionaries, should be interpreted as having a meaning that is consistent with their meaning in the context of the relevant art and should not be interpreted in an idealized and/or overly formal sense unless expressly so defined herein.

Furthermore, relative terms such as “below,” “lower,” “above,” and “upper” may be used herein to describe one element's relationship to another element as illustrated in the accompanying drawings. Such relative terms are intended to encompass different orientations of illustrated technologies in addition to the orientation depicted in the accompanying drawings. For example, if a device in the accompanying drawings is turned over, then the elements described as being on the “lower” side of other elements would then be oriented on “upper” sides of the other elements. Similarly, if the device in one of the figures is turned over, elements described as “below” or “beneath” other elements would then be oriented “above” the other elements. Therefore, the example terms “below” and “lower” can, therefore, encompass both an orientation of above and below.

In this description, for purposes of explanation and not limitation, specific details are set forth, such as particular embodiments, procedures, techniques, etc. in order to provide a thorough understanding of the present invention. However, it will be apparent to one skilled in the art that the present invention may be practiced in other embodiments that depart from these specific details.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” or “according to one embodiment” (or other phrases having similar import) at various places throughout this specification are not necessarily all referring to the same embodiment. Furthermore, the particular features, structures, or characteristics may be combined in any suitable manner in one or more embodiments. Furthermore, depending on the context of discussion herein, a singular term may include its plural forms and a plural term may include its singular form.

What is claimed is:

1. A device for use with a ventilator, the device comprising:
a housing comprising a breath capture module that collects a breath sample from a patient;
an input in fluid communication with an expiratory limb of a ventilator and the breath capture module, the input receiving a breath of the patient;
an output in fluid communication with the expiratory limb of a ventilator to return the breath to the ventilator;
a pump; and
a controller comprising a processor and memory for storing instructions, the processor executing the instructions to control the pump to obtain the breath sample by drawing the breath into the input, across the breath capture module, and out of the output.

2. The device according to claim 1, further comprising a first conduit that couples the expiratory limb to the input, and a second conduit that couples the output to the expiratory limb at a location that is proximate to the ventilator and downstream from the first conduit.

3. The device according to claim 2, wherein the first conduit is coupled with a fitting that is installed inline on the expiratory limb.

4. The device according to claim 1, wherein the processor is configured to obtain a predetermined number of breath samples, the predetermined number of breath samples being based on a flowrate or a periodicity of the breath.

5. The device according to claim 1, wherein when the breath contacts the breath capture module, contents of the breath are captured by the breath capture module.

6. The device according to claim 1, further comprising a first valve associated with the input and a second valve associated with the output, the first valve and the second valve being used to seal the breath capture module.

7. The device according to claim 6, wherein when the first valve and the second valve are sealed, the breath capture module can be removed and replaced.

8. The device according to claim 6, wherein the processor is configured to:
continuously operate the pump;
monitor inspiratory phases and expiratory phases of the ventilator; and
open or close the pump in response to expiratory phases.

9. The device according to claim 1, wherein the processor is configured to control a flow rate of the pump in response to a signal received from the ventilator.

10. The device according to claim 9, wherein the signal is indicative of an operational voltage of the ventilator, the operational voltage ranging from a first voltage value that is indicative of an inspiratory phase and a second voltage value that is indicative of an expiratory phase.

11. The device according to claim 10, wherein the processor is configured to selectively control the pump to turn on in response to detection of a beginning of the expiratory phase and off in response to a beginning of the inspiratory phase or an end of the expiratory phase.

12. A system, comprising:
a ventilator having an inspiratory limb and an expiratory limb;
a breath capture device coupled with the expiratory limb of the ventilator, the breath capture device comprising:
a breath capture module;
a pump; and
a controller comprising a processor and memory for storing instructions, the processor executing the instructions to control the pump to:
obtain breath from the expiratory limb of the ventilator; and
return the breath to the expiratory limb after the breath has contacted the breath capture module, the breath capture module collecting a breath sample from the breath.

13. The system according to claim 12, further comprising a first conduit that couples the expiratory limb to an input of the breath capture device, and a second conduit that couples an output of the breath capture device to the expiratory limb at a location that is proximate to the ventilator and downstream from the first conduit.

14. The system according to claim 12, wherein the processor is configured to obtain a predetermined number of breath samples, the predetermined number of breath samples being based on a flowrate or a periodicity of the breath.

15. The system according to claim 12, wherein the processor is configured to control a flow rate of the pump in response to a signal received from the ventilator, the signal being indicative of an operational voltage of the ventilator, the operational voltage ranging from a first voltage value that is indicative of an inspiratory phase and a second voltage value that is indicative of an expiratory phase.

16. A method, comprising:
determining a flowrate or periodicity of an expiratory phase of a ventilator; and
controlling a pump in response to the flowrate or periodicity to obtain a breath sample, wherein the breath sample is obtained by directing breath from an expiratory limb of the ventilator through a breath capture module and returning the breath to the expiratory limb.

17. The method according to claim 16, wherein controlling the pump comprises controlling a flow rate of the pump in response to a signal received from the ventilator, the signal being indicative of an operational voltage of the ventilator, the operational voltage ranging from a first voltage value that is indicative of an inspiratory phase and a second voltage value that is indicative of the expiratory phase.

18. The method according to claim 16, wherein controlling the pump comprises:
continuously operating the pump at a flow rate;
monitoring an inspiratory phase and the expiratory phase of the ventilator;
opening a first valve of the during the expiratory phase, the first valve being coupled with the expiratory limb and the breath capture module; and
closing the first valve of the during the inspiratory phase.

19. The method according to claim 16, further comprising operating the pump a number of times to obtain a predetermined number of breath samples.

20. The method according to claim 19, wherein the predetermined number of breath samples is based on a flowrate or a periodicity of the breath.
